(19) United States
(12) Patent Application Publication
(10) Pub. No.: US 2010/0119607 A1
(43) Pub. Date: May 13, 2010

(54) BIOENHANCED COMPOSITIONS

(75) Inventors: Nagesh R. PALEPU, South Hampton, PA (US); Pratibha S. PILGAONKAR, Mumbai (IN); Maharukh T. RUSTOMJEE, Mumbai (IN); Anilkumar S. GANDHI, Mumbai (IN); Paras R. JAIN, Mumbai (IN)

Correspondence Address: BLANK ROME LLP, WATERGATE, 600 NEW HAMPSHIRE AVENUE, N.W., WASHINGTON, DC, 20037, US

(73) Assignee: Rubicon Research Pvt. Ltd.

(21) Appl. No.: 12/557,181

(22) Filed: Sep. 10, 2009

Related U.S. Application Data
(63) Continuation of application No. 11/918,728, now abandoned.

(30) Foreign Application Priority Data
Apr. 18, 2005 (IN) 477/MUM/2005
Mar. 6, 2006 (IN) 0315/MUM/2006

(51) Int. Cl. A61K 31/41; A61K 9/10; A61K 31/4184; A61K 31/4178; A61P 3/10; A61P 25/22; A61P 13/12; A61P 25/28; A61P 25/24; A61P 3/04

(52) U.S. Cl. 424/487; 424/484; 424/486; 424/488; 514/381; 514/382; 514/394; 514/397

(57) ABSTRACT

The present invention relates to the method of increasing the bioavailability of Angiotensin II Receptor Blockers (ARBs) by preparing a composition of an ARB with at least one solubility enhancing agent. The invention is particularly focused to provide a novel or modified dissolution profile where the release of ARB in the GI tract is independent of physiological pH conditions.

This application claims the priority of Indian Patent Application Nos. 477/MUM/2005, filed Apr. 18, 2005, and 0315/MUM/2006, filed Mar. 6, 2006, the disclosures of which are incorporated herein by reference.

### FIELD OF THE INVENTION

The present invention relates to the method of increasing the bioavailability of Angiotensin II Receptor Blockers (ARBs) by preparing a composition of an ARB with at least one solubility enhancing agent. The invention is particularly focused to provide a novel or modified dissolution profile where the release of ARB in the GI tract is independent of physiological pH conditions.

### BACKGROUND OF THE INVENTION

Angiotensin II is a very potent end product chemical that causes the muscles surrounding the blood vessels to contract, which thereby significantly narrowing the blood vessels. This narrowing increases the pressure within arterial vessels, causing high Wood pressure (hypertension). Angiotensin receptor blockers (ARBs) are drugs that block the action of angiotensin II. As a result, arterial vessels dilate and blood pressure is reduced, thereby making it easier for the heart to pump blood. ARBs can therefore also be used to improve heart failure as well as hypertension. In addition, they slow the progression of kidney disease due to high blood pressure or diabetes.

The importance of aggressive blood pressure control is undisputed, but the therapeutic focus is now extending to end-organ protection as a treatment goal of equal importance to BP reduction. Thus, the value of ARBs in slowing the progression of kidney disease due to high blood pressure or diabetes has very positive medical as well as commercial implications.

Drugs in this class include candesartan (Atacand, Astra-Zeneca), eprosartan (Teveten, Solvay & Biovail), irbesartan (Avapro, BMS), losartan (Cozaar, Merck), olmesartan (Benicar, Medoxomil; Sankyo & Forest), telmisartan (Micardis, Boehringer Ingelheim), valsartan (Diovan, Novartis) and pratosartan (Kotobuki). ARBs are used alone or in combination with other classes of antihypertensive agents that include thiazide diuretics, β-blockers, calcium channel blockers, rennin inhibitor, and ACE inhibitors, both for the treatment of hypertension and congestive heart failure.

Valsartan, a selective ARB, is a well-known antihypertensive agent. Valsartan is rapidly absorbed from the gastrointestinal tract after oral administration. The absolute bioavailability of valsartan is about 25% (10-35%). This relatively low bioavailability of valsartan is primarily due to its poor solubility in the acid milieu of the gastrointestinal tract. Valsartan is an acid, and therefore, has good solubility at pH>5 and low solubility in acidic conditions of the gastrointestinal (GI) milieu. Valsartan is absorbed from the small intestine where its solubility is low.

The synthesis and use of valsartan are described in U.S. Pat. No. 5,399,578 ('578 patent). Various polymorphs and salt forms of valsartan are described by WO 04083192, WO04087681, and WO03066606.

WO 01401535 relates to pharmaceutical compositions and a method of reducing the risk of morbidity and mortality in patients having symptomatic heart failure comprising administering to such patient an effective amount of valsartan, or pharmaceutically acceptable salts thereof, alone or in combination with another therapeutic agent, optionally in the presence of a pharmaceutically acceptable carrier. This patent describes the novel use or novel crystalline forms of valsartan, but does not tackle the problem associated with the poor bioavailability of valsartan.

U.S. Pat. No. 6,294,197 ('197 patent) and US Patent Application Publication No. 2003/0035832 describe the solid oral dosage forms of valsartan alone or in combination with hydrochlorothiazide (HCTZ) along with a pharmaceutical additive for the preparation of solid dosage forms by a compression method. The solid dosage form according to this invention contains more than 35% by weight of the active agent in the formulation. A process of making such dosage form employing roll compaction is also disclosed.

In U.S. Pat. No. 6,485,745 (745), solid dosage forms of valsartan are described which exhibit accelerated release of the active agent in pH 6.8 phosphate buffer. However release in 0.1 N HCl is not addressed where the solubility of valsartan is minimal.

US Patent Application Publication No. 2002/0132839 and US Patent Application Publication No. 2003/0152620 discuss pharmaceutical compositions of valsartan tablet dosage form are at least 1.2 times more bioavailable than the conventional valsartan capsule. The tablet formulation according to the invention contains a disintegrant at concentration level of 10-80% based on total weight of the composition. The higher amount of disintegrant ensures that the hydrophobic valsartan is wetted well during the granulation stage. The tablet is readily dispersed as granules in the dissolution medium resulting in a better dissolution and improved bioavailability over the normal formulation. The invention does not, however, describe methods to increase solubility of the valsartan itself in the gastric milieu; and therefore, the dissolution of valsartan in 0.1N HCl still remains low which results in low bioavailability.

Candesartan cilexitil, like valsartan, is a hydrophobic molecule with poor aqueous solubility resulting in poor oral availability (about 14%). WO 2005/070398 A2 claims pharmaceutical compositions in the form of tablets that include candesartan cilexitil, fatty acid glycerides, a surfactant, a co-solvent and pharmaceutically acceptable additives. The formulation is further coated with a film forming polymer and polyethylene glycol. The co-solvent employed only improves the stability of candesartan and does not alter its solubility or dissolution rate in acidic medium.

United States Patent Application Publication No. US 2005/0220881 provide methods of improving dissolution of Eprosartan by preparing its association complex with one or more solid poloxamers. However, a large amount of poloxamers is required to achieve significant dissolution enhancement. The dosage form development of such a complex that would achieve a higher oral bioavailability becomes very difficult due to weight limitations. Moreover, large amount of poloxamers for chronic use may not be allowed.

The low bioavailability associated with poor aqueous solubility warrants administration of larger doses of the ARBs to maintain desired therapeutic activity. Thus there remains a need and opportunity for an improved ARB formulation that delivers the active form of medicament both in the solubilized form and in a predictable manner over the wider pH range of the GI tract.

### SUMMARY OF INVENTION

The present invention relates to methods of predictably increasing the bioavailability of ARBs, especially valsartan, and insuring consistent absorption over a wide pH range of the GI tract. This invention increases the absorption for ARBs in the GI tract and thereby reduces inter- and intra-patient variability, which is in contrast to the current marketed oral dosage formulations which have highly variable intra- and inter-patient absorption. This invention also leads to a significant decrease in the time to reach maximum blood concentration (Tₘₐₓ) and extent of absorption (AUC) of an ARB compared to the marketed product.

The invention also relates to a physically and chemically stable formulation of ARBs, in particular, valsartan, utilizing generally recognized as safe (GRAS) excipients. This invention also relates to an oral dosage formulation of valsartan having reduced intra- and inter-patient variability in absorption, particularly at low GI pH. The coefficient of variability for Cₘₐₓ, the maximum concentration in the blood, is less than about 35%, preferably less than about 30%. The coefficient of variability for the AUC is less than about 45%, preferably 40%, and most preferably 30%.

It has been discovered that an ARB, particularly valsartan, when combined with a solubility enhancing agent significantly increases its solubility in acidic environment (pH<3) as well as an improved dissolution rate which is in sharp contrast to the currently marketed ARB formulations. The bioavailability will also increase as more drug is present in the solubilized form at the absorption site. The increase in bioavailability reduces the dose of the ARB required to achieve the desired effect as well as patient to patient variability, and thus, enhances the therapeutic utility of the ARB. The solid dosage form can be manufactured using conventional manufacturing processes and standard processing equipments that are generally used to manufacture the solid dosage form.

In one aspect, the present invention provides a method of increasing the bioavailability of an ARB by administering it with at least one solubility enhancing agent.

In yet another aspect, this novel composition of valsartan provides at least 40% dissolution in acidic and weakly acidic dissolution medium. “Acidic” as used herein refers to pH less than about 3. “Weakly acidic” as used herein refers to pH of about 3 to 5.

The composition of the present invention of valsartan may be used to treat the diseases described below and to deliver the solubilized form of the drug over the wide pH range of the GI tract to increase bioavailability. Therefore, the dose and frequency of administration can be reduced, compared with administration of conventional valsartan. Moreover, the inter- and intra-patient variability associated with the current formulation of valsartan can also be reduced. Therefore, it is expected that there will be an increased therapeutic effect from this composition of the present invention of valsartan.

Examples of the diseases to be treated by this agent include 1. circulatory disease, such as hypertension, cardiac disease (heart failure, myocardial infarction, valvular disease), peripheral circulatory insufficiency; 2. kidney disease, e.g., glomerulonephritis, renal insufficiency; 3. cerebral dysfunction, e.g., stroke, Alzheimer's disease, depression, amnesia, dementia; 4. diabetic complications, e.g., retinopathy, nephropathy; 5. arteriosclerosis manifested by hypertension, stroke, heart attack, angina, or ischemia of gastrointestinal (GI) tract or extremities; 6. unique conditions, e.g.; hyperaldosteronism, multiple system organ failure, scleroderma; and 7. anxiety neurosis, catatonia, and dyspepsia. Many of these conditions are caused or exacerbated by vasoconstriction expressed secondary to angiotensin II.

### BRIEF DESCRIPTION OF THE DRAWINGS

FIG. 1 is a graph comparing dissolution rates of valsartan by itself and valsartan with solubility enhancing agent as a solid dispersion;

FIG. 2 is a graph showing in vitro release profiles of Diovan®. formulation #1 of example 4.

FIG. 3 is a graph showing in vitro release profiles of the valsartan formulation #2 of example 6.

FIG. 4 is a graph showing in vitro release profiles of the valsartan formulation #3 of example 7.

FIG. 5 is a graph comparing plasma profiles of the valsartan formulation #4 of example 8 and Diovan®.

FIG. 6 is a graph comparing dissolution profiles of the valsartan formulation at different pH.

### DETAILED DESCRIPTION OF THE PREFERRED EMBODIMENTS

According to the basic principle of drug absorption, only the drug in the neutral form present in solution can permeate across the lipid cell membranes. Therefore, it is very essential for a better absorption, the drug substance should be lipophilic in nature and have adequate solubility in the GI milieu. In general, chemically, most of ARBs have a common feature, i.e., at least one free carboxylic acid group, which makes ARBs insoluble in acid conditions and ionized (soluble form) in alkaline environment. For example, valsartan has a carboxylic acid group, and therefore, it is not readily soluble in acidic medium. Absorption of valsartan in an acidic environment is, therefore, low due to its poor solubility. However, in an alkaline environment valsartan is in the ionized form which is not as lipophilic as the neutral free acid and thus permeates poorly through the cell membranes. In other words, valsartan has poor absorption in the gastrointestinal tract either due to a combination of poor solubility of the free acid form in acidic/weakly acidic GI milieu and poor permeability of the dissolved (ionized) form. The result is low bioavailability of 10-25%. Even those ARBs which do not possess any carboxylic acid functionality exhibit low solubility in acidic/weakly acid medium.

Ionization at higher pH is the intrinsic property of the ARB molecule itself and it is not possible to alter this parameter. The only possibility that remains is to increase the solubility and/or dissolution rate in the acidic environment. It has been surprisingly found that a combination of an ARB or its salt in the presence of certain substances, which are referred to herein as solubility enhancing agent, results in increased solubility and improved dissolution rate over a wider pH range leading to an improved bioavailability compared to the marketed presentation.

In addition, the chemical substances having free carboxylic acid groups, such as ARBs, are generally regarded as substrates for the p-glycoprotein reverse transport system. This p-glycoprotein mediated efflux is also in part responsible for the reduced bioavailability of ARBs. The solubility enhancing agent employed in the present application may also acts as inhibitor of p-glycoprotein which helps to further increase in the bioavailability of the ARB.

### Active Ingredient

The active ingredient for the purpose of this invention is an ARB, which may be, but not limited to, candesartan, eprosartan, irbesartan, losartan, olmesartan, telmisartan, valsartan, or pratosartan. The active ingredient of the invention may be present in crystalline or amorphous form. The crystalline form may have different polymorphs. All different polymorphs, solvates, hydrates, salts are within the purview of this invention. The preferred active ingredient is valsartan.

In the dosage form of the present invention, in addition to an ARB, one or more, for example two or three, active ingredients may be combined. The therapeutic agents, which may be combined with an ARB include, but are not limited to, anti-hypertensive agents, such as hydrochlorothiazide (HCTZ), calcium blockers, beta-blockers, ACE inhibitors, inotropic agents, hypolipidemic agents, anti-obesity agents, rennin inhibitors, and/or anti-diabetic agents.

The present invention is also applicable to other active pharmaceutical ingredients having similar low solubility related bioavailability issues.

The active ingredient may be present in an amount of about 1-80%, preferably 5-50%, and more preferably 10-30% by weight of the composition.

### Solubility Enhancing Agent

According to this invention, the increase in instantaneous solubility of an ARB in a composition is achieved by using one or more suitable solubility enhancing agent. The solubility enhancing agent may include one or more surfactant, solubilizer, complexing agent, hydrotropic agent, and the like. The solubility enhancing agent could be the same or different for different ARB's. This invention is particularly focused to provide a novel or modified dissolution profile where the release of ARB is similar throughout physiological pH conditions of the gastrointestinal tract, i.e. the drug release is substantially independent of physiological pH conditions.

The solubility enhancing agent may be, but not limited to, hydrophilic surfactants, lipophilic surfactants, mixtures there of. The surfactants may be anionic, nonionic, cationic, zwitterionic or amphiphilic. The relative hydrophilicity and hydrophobicity of surfactants is described by HLB (hydrophilic-lipophilic balance) value. Hydrophilic surfactants include surfactants with HLB greater than 10 as well as anionic, cationic, amphiphilic or zwitterionic surfactants for which the HLB scale is not generally applicable. Similarly, lipophilic surfactants are surfactants having an HLB value less than 10.

The hydrophilic non-ionic surfactants may be, but not limited to, polyethylene glycol sorbitan fatty acid esters, polyethylene glycol fatty acid monoesters, PEG-fatty acid diesters, hydrophilic trans-esterification products of alcohols or polyols with at least one member of the group consisting of natural and/or hydrogenated oils. The most commonly used oils are castor oil or hydrogenated castor oil, or an edible vegetable oil such as corn oil, olive oil, peanut oil, palm kernel oil, almond oil. Preferred polyols include glycerol, propylene glycol, ethylene glycol, polyethylene glycol, sorbitol and pentaerythritol. Preferred hydrophilic surfactants in this class include PEG-35 castor oil, polyoxyethylene-polypropylene copolymer (Lutrol, BASF), and PEG-40 hydrogenated castor oil.

The amphiphilic surfactants includes, but are not limited to, d-α-tocopheryl polyethylene glycol 1000 succinate and d-α-tocopherol acid salts such as succinate, acetate, etc.

The ionic surfactants may be, but not limited to, alkylammonium salts; fusidic acid salts; fatty acid derivatives of amino acids, oligopeptides, or polypeptides; glyceride derivatives of amino acids, oligopeptides, or polypeptides; lecithins or hydrogenated lecithins; lysolecithins or hydrogenated lysolecithins; phospholipids or derivatives thereof; lysophospholipids or derivatives thereof; carnitine fatty acid ester salts; salts of alkylsulfates; fatty acid salts; sodium docusate; acyl lactylates; mono- or di-acetylated tartaric acid esters of mono- or di-glycerides; succinylated mono- or di-glycerides; citric acid esters of mono- or di-glycerides; or mixtures thereof.

The lipophilic surfactants may be, but not limited to, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols or sterol derivatives; polyethylene glycol alkyl ethers; sugar esters; sugar ethers; lactic acid derivatives of mono- or di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; polyethylene glycol (PEG) sorbitan fatty acid esters; PEG glycerol fatty acid esters; polyglycerized fatty acid; polyoxyethylene-polyoxypropylene block copolymers; sorbitan fatty acid esters; or mixtures thereof.

Preferably, the solubility enhancing agent may be PEG-20-glyceryl stearate (Capmul® by Abitec), PEG-40 hydrogenated castor oil (Cremophor RH 40® by BASF), PEG 6 corn oil (Labrafil® by Gattefosse), lauryl macrogol—32 glyceride (Gelucire 44/14® by Gattefosse), stearoyl macrogol glyceride (Gelucire 50/13® by Gattefosse), polyglyceryl-10 mono dioleate (Caprol® PEG 860 by Abitec), propylene glycol oleate (Lutrol OP® by BASF), propylene glycol dioctanoate (Captex® by Abitec), propylene glycol caprylate/caprate (Labrafac® by Gattefosse), glyceryl monooleate (Peceol® by Gattefosse), glycerol monolinoleate (Maisine® by Gattefosse), glycerol monostearate (Capmul® by Abitec), PEG-20 sorbitan monolaurate (Tween 20® by ICI), PEG-4 lauryl ether (Brij 30® by ICI), sucrose distearate (Sucroester 7® by Gattefosse), sucrose monopalmitate (Sucroester 15® by Gattefosse), polyoxyethylene-polyoxypropylene block copolymer (Lutrol®series BASF), polyethylene glycol 660 hydroxystearate, (Solutol® by BASF), sodium lauryl sulphate, sodium dodecyl sulphate, dioctyl suphosuccinate, L-hydroxypropyl cellulose, hydroxylethylcellulose, hydroxy propylcellulose, propylene glycol alginate, sodium taurocholate, sodium glycocholate, sodium deoxycholate, betains, polyethylene glycol (Carbowax® by DOW), d-α-tocopheryl polyethylene glycol 1000 succinate (Vitamin E TPGS® by Eastman), or mixtures thereof.

More preferably, the solubility enhancing agent may be PEG-40 hydrogenated castor oil (Cremophor RH 40® by BASF—HLB-13), lauryl macrogol-32 glyceride (Gelucire 44/14® by Gattefosse—HLB-14) stearoyl macrogol glyceride (Gelucire 50113® by Gattefosse—HLB-13), PEG-20 sorbitan monolaurate (Tween 20® by ICI—HLB-17), PEG-4 lauryl ether (Brij 30® by ICI—HLB-9.7), polyoxyethylene-polyoxypropylene block copolymer (Lutrol® series BASF having different HLB ranging from 15-30), Sodium lauryl sulphate (HLB-40), polyethylene glycol (Carbowax® by DOW), d-α-tocopheryl polyethylene glycol 1000 succinate (Vitamin E TPGS® by Eastman—HLB-15), or mixtures thereof.

The solubilizers may also include pH modifiers such as buffers, amino acids and amino acid sugars.

The complexing agent includes cyclodextrin class of molecules, such as cyclodextrins containing from six to twelve glucose units, especially, alpha-cyclodextrin, beta-cyclodextrin, gamma-cyclodextrin, or their derivatives, such as hydroxypropyl beta cyclodextrins, or mixtures thereof. The complexing agents may also include cyclic amides, hydroxyl benzoic acid derivatives as well as gentistic acid. In this complexation process, a hydrophilic polymer may be additionally added to further enhance the solubility along with the complexing agent.

In the composition of the present invention, the ARB and one or more solubility enhancing agents may be employed in different ratios. The selected ratio depends upon the desired improvement in solubility and the type of solubility enhancing agents employed. It is contemplated within the scope of the invention that the ratio of ARB to solubility enhancing agents may range from about 20:1 to about 1:20, preferably from about 10:1 to about 1:10, and most preferably about 5:1 to about 1:5. A combination of solubility enhancing agents may also be included where the total amount of solubility enhancing agent employed is maintained in the above-mentioned ratios.

### Solubilization of ARBs

In the composition, the ARB may be present in the form of physical blend, solid dispersion, solid solution or complex with the solubility enhancing agent. Different processes may be employed to prepare the composition of the ARB with the solubility enhancing agents. It is contemplated within the scope of the invention that the processes may include, but not limited to, solubilization using melt granulation, solvent treatment, physical mixing or spray drying of the dissolved in a solvent with a solubility enhancing agent.

In the case of melt granulation, the solubility enhancing agent is melted. The ARB is then added and mixed with the molten mass, and allowed to solidify to form granules which are then separated from each other. In another illustrative embodiment of this system, the ARB is granulated using a molten solubility enhancing agent. In some cases, the ARB and the solubility enhancing agent both may be melted together and congealed to room temperature.

In using a solvent treatment method, either the solubility enhancing agents or the ARB, or both, are dissolved in a solvent which is then evaporated or spray dried. The resultant mass is a blend of ARB and solubility enhancing agent, such that the solubility of the ARB is increased. The solvent employed in this system may be aqueous or non-aqueous.

In the case of physical mixing, the ARB and the solubility enhancing agent are preferably intimately dry-mixed using a Hobart mixture, a V-blender, or a high shear granulator.

In the complexation method, complex of ARB can be prepared using different techniques such as ball milling, solvent evaporation method which includes spray drying and lyophilization process, slurry method, paste method, etc.

It is contemplated within the scope of the invention that a combination of aforementioned processes can be employed. For example, a combination of hot melt process, physical mixing, and solvent treatment method may be employed. In this case, the ARB may be initially granulated with one or more molten solubility enhancing agents, which can be further treated with the same or different solubility enhancing agents in a solvent or with simple physical mixing or vice versa. It is also contemplated within the scope of the invention that any process known in the art suitable for making pharmaceutical compositions in general may be employed for the purpose of this invention.

Melt granulation and intimate physical mixture are the most preferred methods for preparing valsartan according to the present invention. The increase in solubility may be determined by studying the actual solubility studies of the valsartan in presence of the solubility enhancing agent, or by carrying out dissolution studies in an appropriate dissolution medium. The dissolution method is preferred, as it allows the comparison of the rate of dissolution of different formulations by determining the amount of valsartan dissolved at different time intervals.

The composition may be incorporated in various pharmaceutical dosage forms, including, but not limited to, tablets which disintegrate in stomach, tablets which can disintegrate in the mouth, tablets which can disintegrate by effervescence in a liquid (water), tablets which can be dispersed in a liquid (such as water), coated tablets, powders of given doses packaged in sachets, suspensions, gelatin capsules, soft gelatin capsules, semisolid dosage forms, and other drug delivery systems.

The preferred dosage form of the present invention is a solid dosage form, preferably a tablet, which may vary in shape, including, but not limited to, oval, triangle, almond, peanut, parallelogram, pentagonal. It is contemplated within the scope of the invention that the dosage form could be encapsulated.

Tablets in accordance with the invention may be manufactured using conventional techniques of tableting known in the art, such as, but not limited to, direct compression, wet granulation, dry granulation, or extrusion/melt granulation.

The dosage form according to the invention may include excipients conventionally known in art such as fillers, binders and lubricants. Fillers, such as, but not limited to, lactose monohydrate, microcrystalline cellulose, dicalcium phosphate, calcium silicate, magnesium aluminometasilicate (Neusillin), or the like, may be used. Binders, such as, but not limited to, polyvinyl pyrrolidone (PVP), copovidone, or the like, may be used. Lubricants, such as, but not limited to, Aerosil-200, magnesium stearate, hydrogenated vegetable oils, triglycerides of stearic acid, palmitic acid, or the like, may be utilized.

The disintegrating agent may be, but not limited to, the following: starch, sodium starch glycolate, pregelatinised starch, crosslinked poly vinyl pyrrolidone, cross linked carboxy methyl cellulose, or ion exchange resin, most preferrably sodium starch glycolate. The disintegrant may be present in an amount ranging from about 0.25% to about 30%, more preferably about 0.5 to about 20.0% and most preferably about 0.75-10% by weight based on the total weight of the composition.

In one illustrative embodiment, the dosage form may optionally be coated. Surface coatings may be employed for aesthetic purposes or for dimensionally stabilizing the compressed dosage form. The surface coating may be carried out using any conventional coating agent which is suitable for oral use. The coating may be carried out using any conventional technique employing conventional ingredients. A surface coating may, for example, be obtained using a quick-dissolving film using conventional polymers, such as hydroxypropyl methyl cellulose, hydroxypropyl cellulose, carboxymethyl cellulose, polyvinyl alcohol poly methacrylates, or the like.

In one illustrative embodiment, the solubilized ARB may be incorporated in liquid form into a capsule. In this embodiment, the ARB mixed with a molten solubility enhancing agent is filled into capsules with or without other excipients. The content of the capsule may remain in liquid or semisolid state during shelf life or the liquid filled into the capsule may set to form a solid mass inside capsule. Optionally excipients, such as disintegrants, lubricants, or diluents, may be included in the formulation.

In another illustrative embodiment, the solubilized ARB may be dispersed in an excipient, such as microcrystalline cellulose, lactose, mannitol, calcium silicate, magnesium aluminometasilicate (Neusillin) or any other excipient that is generally employed in oral dosage forms. The dispersed mixture can be filled into a capsule or compressed into a tablet.

In another illustrative embodiment, the solubilized ARB may be incorporated into a sustained release formulation. The solubility enhancing agent ensures better control over the release profile and also complete release of the drug in the desired time interval.

In a further illustrative embodiment, the solubilized ARB may be incorporated into a sustained release matrix formulation comprising one or more polymeric or non-polymeric release retardants. Examples of polymers that may be used include, but are not limited to, polyalkylene oxides; cellulosic polymers; acrylic acid, methacrylic acid polymers, and esters thereof; maleic anhydride polymers; polymaleic acid; poly(acrylamides); poly(olefinic alcohols; poly(N-vinyl lactams); polyols; polyoxyethylated saccharides; polyoxazolines; polyvinylamines; polyvinylacetates; polyimines; starch and starch-based polymers; polyurethane hydrogels; chitosan; polysaccharide gums; zein; shellac-based polymers; polyethylene oxide; hydroxypropyl cellulose; hydroxypropyl methyl cellulose; hydroxyethyl cellulose; sodium carboxy methylcellulose; calcium carboxymethyl cellulose; methyl cellulose; polyacrylic acid; maltodextrin; pre-gelatinized starch and polyvinyl alcohol; copolymers; and mixtures thereof.

One or more hydrophilic polymers may also be used to prepare sustained release dosage forms. These polymers are preferably polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch, polyvinyl alcohol, or mixtures thereof. The weight percent of the hydrophilic polymer in the dosage form is about 5 to about 90 weight percent, preferably about 10 to about 70 weight percent, and most preferably about 15 to about 50 weight percent.

In a further illustrative embodiment a solid pharmaceutical composition may be in the form of a multilayer system for oral administration. The system may be adapted to deliver two different actives such as solubilized ARB in one layer and hydrochlorothiazide in another layer.

In a further illustrative embodiment, a solid pharmaceutical composition in the form of a multilayer system for oral administration may be adapted to deliver a first active pharmaceutical agent from a first layer immediately upon reaching the gastrointestinal tract, and to deliver a second pharmaceutical agent, which may be same or different from the first agent, from a second layer, in a controlled manner over a specific time period.

In a further illustrative embodiment, a solid pharmaceutical composition in the form of an expanding multilayer system for oral administration is adapted to deliver a first active pharmaceutical agent from a first layer immediately upon reaching the gastrointestinal tract, and to deliver a second pharmaceutical agent, which may be same or different from the first agent, from a second layer, in a controlled manner over a specific time period. The second layer is also adapted to provide expanding nature for the dosage system, thereby making the dosage system have greater retention in the stomach.

In another illustrative embodiment, the solubilized ARB may be incorporated into an osmotically controlled drug delivery system. The solubility enhancing agent ensures better control over the release profile and also complete release of the drug in the desired time interval.

Solubility enhancing agents used in the composition will increase solubility and dissolution of ARBs, particularly valsartan, in acidic and weakly acidic environment. Immediate release dosage form comprising the composition provides at least 40% dissolution in an acidic and weakly acidic environment. The solubility enhancement and a higher dissolution in acidic and weakly acidic environment result in more drug permeating through the GI membrane that leads to increased bioavailability. This increase in solubility also results in a pH independent drug release profile for a drug that is having pH dependent solubility. This invention also reduces inter- and intra-patient variability in drug absorption.

The present invention provides oral solid dosage forms of ARBs that are about 1.2 to 4 times more bioavailable than the conventional immediate release dosage forms. The increase in bioavailability is evident from the decrease in Tₘₐₓ (time to reach maximum blood concentration), and the increase in Cₘₐₓ (the maximum blood concentration), AUC₀₋ₜ, and AUC_{0-∞} (the extent of absorption, or area under the blood concentration v. time curve). Due to the increase in relative bioavailability, the novel composition will also be able to reduce the variability typically associated with an ARB, especially where the ARB is valsartan. This composition may also achieve peak plasma concentration in less than 4 hours, preferably in less than 3 hours, and more preferably in less than 2 hours. The achieved Tₘₐₓ value is also faster than the conventional immediate release formulation.

While the present invention has been described in terms of its specific illustrative embodiments, certain modifications and equivalents will be apparent to those skilled in the art and are intended to be included within the scope of the present invention. The details of the invention, its objects and advantages are explained hereunder in greater detail in relation to non-limiting exemplary illustrations.

### Example 1

### Solubilization of Valsartan

Solid dispersions of valsartan with different solubility enhancing agents in different ratios were prepared by adding valsartan to the molten mass under continuous mixing to get uniform dispersion. The solubility of the resulting solid dispersion was determined in 0.1N HCl.

TABLE 1
Solubility of valsartan with different
solubility enhancing agents in 0.1N HCl

| | HLB of | |
|---|---|---|
| | solubility | |
| | enhancing | Solubility in |
| Solid dispersion | agent | mcg/ml |
| Valsartan | | 84.60 |
| Valsartan:Stearoyl Macrogol Glycerides, | 13 | 73.51* |
| USP (Gelucire 50/13) 1:0.5 | | |
| Valsartan:Vitamin E T.P.G.S., USP/NF | 15 | 230.40 |
| 1:0.5 | | |
| Valsartan:Vitamin E T.P.G.S., USP/NF | 15 | 337.10 |
| 1:1 | | |
| Valsartan:Stearoyl Macrogol Glycerides, | 13 | 167.19 |
| USP (Gelucire 50/13) 1:1 | | |
| Valsartan:Polyoxyl 40 hydrogenated | 13 | 181.24 |
| Castor oil, USP (Cremophor RH40) 1:1 | | |
| Valsartan:Polyethylene glycol 6000, USP | | 82.54* |
| 1:1 | | |

T.P.G.S. - α-Tocopheryl polyethylene glycol 1000 succinate
HLB - hydrophilic-lipophilic balance
*Results might be underestimated due to assay interference

Among different solubility enhancing agents studied maximum increase in solubility was achieved with vitamin E TPGS, Gelucire 50/13 at 1:1 ratio and in cremophor RH 40.

### Example 2

### Solubilization of Valsartan Using Combination of Surfactants

Solid dispersions of valsartan with different combinations of solubility enhancing agents were prepared by adding valsartan to the molten mass of combination of surfactants under continuous mixing to get uniform dispersion. The solubility of the resulting solid dispersion was determined in 0.1N HCl.

TABLE 2
Solubility of valsartan with different combination
of solubility enhancing agents in 0.1N HCl

| | HLB of the | |
|---|---|---|
| | combination of | |
| | solubility | |
| | enhancing | Solubility in |
| Solid Dispersion | agent | mcg/ml |
| Valsartan | | 84.60 |
| Valsartan:Stearoyl Macrogol Glycerides, | 17.5 | 140 |
| USP (Gelucire50/13):SLS*, USP | | |
| 1:0.5:0.1 | | |
| Valsartan:Stearoyl Macrogol Glycerides, | 15.5 | 171.19 |
| USP (Gelucire50/13):SLS, USP | | |
| 1:1:0.1 | | |
| Valsartan:Polyoxyl 40 hydrogenated | 17.5 | 123.7 |
| castor oil, USP (Cremophor RH40):SLS, | | |
| USP 1:0.5:0.1 | | |
| Valsartan:Polyoxyl 40 hydrogenated | 15.5 | 173.9 |
| castor oil, USP (Cremophor RH40):SLS, | | |
| USP 1:1:0.1 | | |

*SLS - Sodium lauryl sulphate

In combination of surfactants maximum increase in solubility was achieved by a combination of Cremophor RH40 and SLS.

### Example 3

### Preparation of Solid Dispersion of Valsartan and Study its Dissolution Rate

Gelucire was melted in a beaker on a hot plate with temperature set at about 50° C. and to the molten mass valsartan was added in the ratio of 1:0.5 (valsartan:Gelucire) and mixed for some time. To this mixture 2 parts microcrystalline cellulose were added and mass stirred till it achieved room temperature. The dissolution was carried out by adding the weighed amount of dispersion (in this case 280 mg) directly to the dissolution jars.

### In-Vitro Dissolution Studies

In-vitro dissolution studies were carried out with following specifications
Dissolution Medium: 0.1N HCL with 0.5% SLS

### Dissolution Test Apparatus: USP Type II

### Temperature: 37.5±0.5° C.

### RPM: 50

Sampling intervals: 5, 10, 15, 30, 60 and 120 minutes
Sampling volume: 10 ml

TABLE 3
In vitro dissolution studies of valsartan
alone and solid dispersion of valsartan

| Time | Valsartan | Valsartan solid dispersion |
|---|---|---|
| (minutes) | % Cum. Dissolved | % Cum. Dissolved |
| 0 | 0 | 0 |
| 5 | 1.0 | 32.2 |
| 10 | 4.1 | 32.9 |
| 15 | 6.7 | 36.6 |
| 30 | 6.5 | 40.6 |
| 60 | 9.7 | 43.7 |
| 120 | 15.7 | 42.7 |

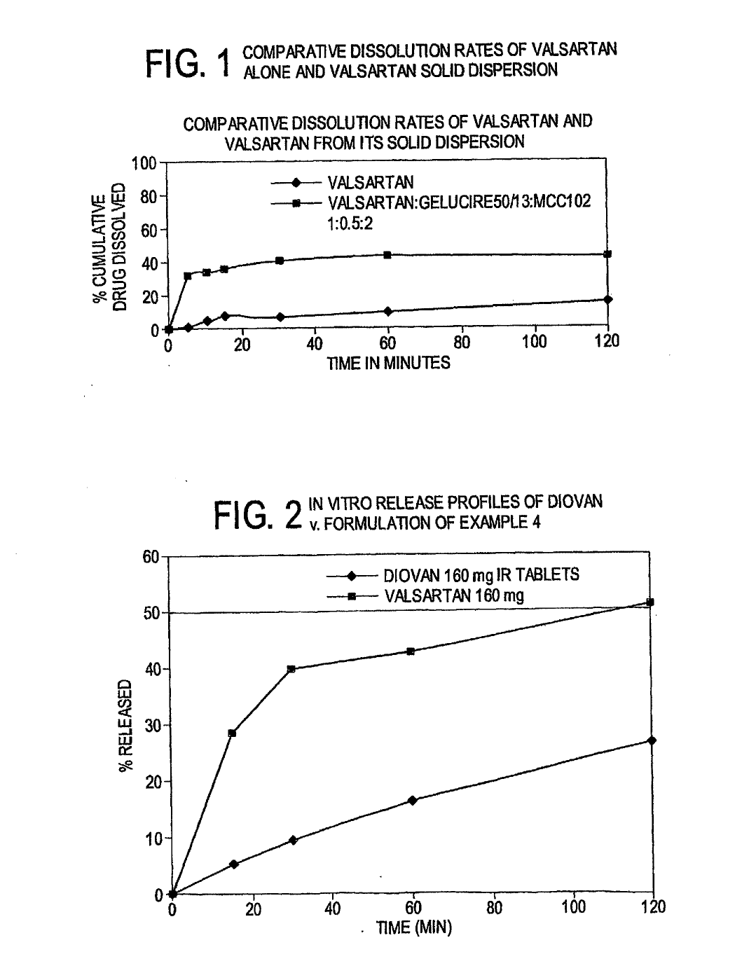

A significant increase in the dissolution rate was achieved for valsartan with a solubility enhancing agent in the solid dispersion. At the end of 120 min only 15% of pure valsartan was dissolved. However in the case of valsartan with the solubility enhancing agent in the solid dispersion, more than 30% of the drug was dissolved in first 5 min. (FIG. 1)

### Example 4

### Incorporation of Solubilized Valsartan into Immediate Release Tablet Formulation (Formulation #1)

TABLE 4
Immediate release formulation of valsartan-BEx (Formulation #1)

| Ingredients | mg/tab |
|---|---|
| Solid dispersion phase | |
| Valsartan | 160 |
| Stearoyl Macrogol Glycerides, USP (Gelucire 50/13) | 80 |
| Microcrystalline cellulose, USP (Avicel PH 102) | 320 |
| External | |
| Microcrystalline cellulose, USP (Avicel PH 200) | 160 |
| Colloidal Silicon Dioxide, USP (Aerosil 200) | 8 |
| Magnesium Stearate, USP | 8 |
| Cros-povidone, USP (Kollidon.CL) | 22.08 |

Solid dispersion of valsartan and solubility enhancing agent was prepared according to the process of example 3. The dispersion was then mixed with all other excipients, lubricated and compressed into tablets.

### In-Vitro Dissolution Rate Studies

In-vitro dissolution rate studies were carried out with following specifications

### Dissolution Test Apparatus: USP Type II

### Temperature: 37.5±0.5° C.

Dissolution Medium Dissolution studies were carried out in 0.1N HCL (900 ml)

### Rpm: 75

Sampling intervals: 15, 30, 60 and 120 minutes
Sampling volume: 10 ml

TABLE 5
Dissolution profile of Formulation
#1 (Table 4) and Diovan in 0.1N HCL

| | Diovan 160 mg IR tablets | Formulation #1 |
|---|---|---|
| Time (min) | (% Cum. Dissolved) | (% Cum. Dissolved) |
| 0 | 0 | 0 |
| 15 | 5.5 | 28.7 |
| 30 | 9.5 | 39.9 |
| 60 | 16.3 | 42.7 |
| 120 | 26.8 | 51.0 |

The formulation of the present invention exhibited much higher and faster drug release profile than that of Diovan. (FIG. 2).

### Example 5

### Incorporation of Solubilized of Valsartan into a Sustained Release Tablet Formulation

TABLE 6
Sustained release tablet composition

| Ingredients | mg/tab |
|---|---|
| Solid dispersion phase | |
| Valsartan | 160.0 |
| Stearoyl Macrogol Glycerides, USP | 80.0 |
| (Gelucire 50/13) | |
| Microcrystalline cellulose, USP (Avicel PH 102) | 320.0 |
| External | |
| Hydroxy propyl methyl cellulose, USP | 50.0 |
| (Methocel K100M) | |
| Lactose Monohydrate, USP | 250.0 |
| Polyvinyl Pyrrolidone, USP | 75.0 |
| Magnesium Stearate, USP | 9.35 |

The solid dispersion of valsartan with Gelucire 50/13 was dry mixed with other excipients except magnesium stearate and granulated with alcoholic solution of polyvinyl pyrrolidone. Granules were dried in fluidized bed dryer, lubricated and compressed into tablets.

### In-Vitro Drug Release Studies

In-vitro drug release studies were carried out with following specifications

### Dissolution Test Apparatus: USP Type I

### Temperature: 37.5±0.5° C.

Dissolution Medium: 0.1N HCL with 0.5% SLS

### RPM: 100

Sampling intervals: 1, 2, 3, 4, 6, 8, 10, 12 and 18 hours
Sampling volume: 10 ml

TABLE 7
In vitro release of valsartan from the sustained release formulation

| Time in hours | % Cumulative Dissolution |
|---|---|
| 0 | 0 |
| 1 | 2.8 |
| 2 | 7.6 |
| 3 | 9.5 |
| 4 | 12.9 |
| 6 | 21.6 |
| 8 | 28.7 |
| 10 | 40.5 |
| 12 | 53.8 |
| 18 | 97.4 |

### Example 6

### Incorporation of Solubilized Valsartan into an Immediate Release Tablet Formulation (Formulation #2)

TABLE 8
Immediate release formulations of Valsartan-BEx (Formulation #2)

| Ingredients | mg/tab |
|---|---|
| Solid dispersion phase | |
| Valsartan | 80.0 |
| Vitamin E TPGS, USP/NF | 80.0 |
| Microcrystalline cellulose, USP (Avicel PH 102) | 240.0 |
| External | |
| Microcrystalline cellulose, USP (Avicel PH200) | 400.0 |
| Colloidal Silicon Dioxide, USP (Aerosil 200) | 18.0 |
| Magnesium Stearate, USP | 6.0 |
| Cros-povidone, USP (Kollidon.CL) | 25.0 |

Solid dispersion of valsartan with Vitamin E TPGS was prepared according to the process of example 3. The solid dispersion was then mixed with all other excipients, lubricated and compressed into tablets.

### In-Vitro Dissolution Rate Studies

In-vitro dissolution rate studies were carried out with following specifications

### Dissolution Test Apparatus: USP Type II

### Temperature: 37.5±0.5° C.

Dissolution Medium Dissolution studies were carried out in 0.1N HCL (900 ml)

### RPM: 75

Sampling intervals: 15, 30, 60 and 120 minutes
Sampling volume: 10 ml

TABLE 9
Dissolution profile of Formulation #2 (Table 8) in 0.1N HCL

| Time (min) | % Cum. dissolution |
|---|---|
| 0 | 0 |
| 15 | 62.6 |
| 30 | 77.6 |
| 60 | 80.3 |
| 120 | 83.6 |

The developed formulation exhibited much higher and much faster drug release profile (FIG. 3) compared to the marketed Diovan tablet as well as other exploratory valsartan formulations, formulations #1 and #3

### Example 7

### Incorporation of Solubilized Valsartan into an Immediate Release Tablet Formulation (Formulation #3)

TABLE 10
Immediate release formulation of Valsartan-BEx (Formulation #3)

| Ingredients | mg/tab |
|---|---|
| Physical mixture | |
| Valsartan | 80.0 |
| Poloxamer 407, USP (LutrolF127) | 80.0 |
| External | |
| Microcrystalline cellulose, USP (Avicel PH102) | 320.0 |
| Colloidal Silicon Dioxide, USP (Aerosil 200) | 14.4 |
| Magnesium Stearate, USP | 9.6 |
| Cros-povidone, USP | 29.0 |

Valsartan was mixed physically with Lutrol F127. This blend was then further mixed with all other excipients, lubricated and compressed into tablets.

### In-Vitro Dissolution Rate Studies

In-vitro dissolution rate studies were carried out with following specifications

### Dissolution Test Apparatus: USP Type II

### Temperature: 37.5±0.5° C.

Dissolution Medium Dissolution studies were carried out in 0.1N HCL (900 ml)

### RPM: 75

Sampling intervals: 15, 30, 60 and 120 minutes
Sampling volume: 10 ml

TABLE 11
Dissolution profile of Formulation #3 in 0.1N HCL

| Time (min) | % Cum. Dissolved |
|---|---|
| 0 | 0 |
| 15 | 38.9 |
| 30 | 51.9 |
| 60 | 58.8 |
| 120 | 65.5 |

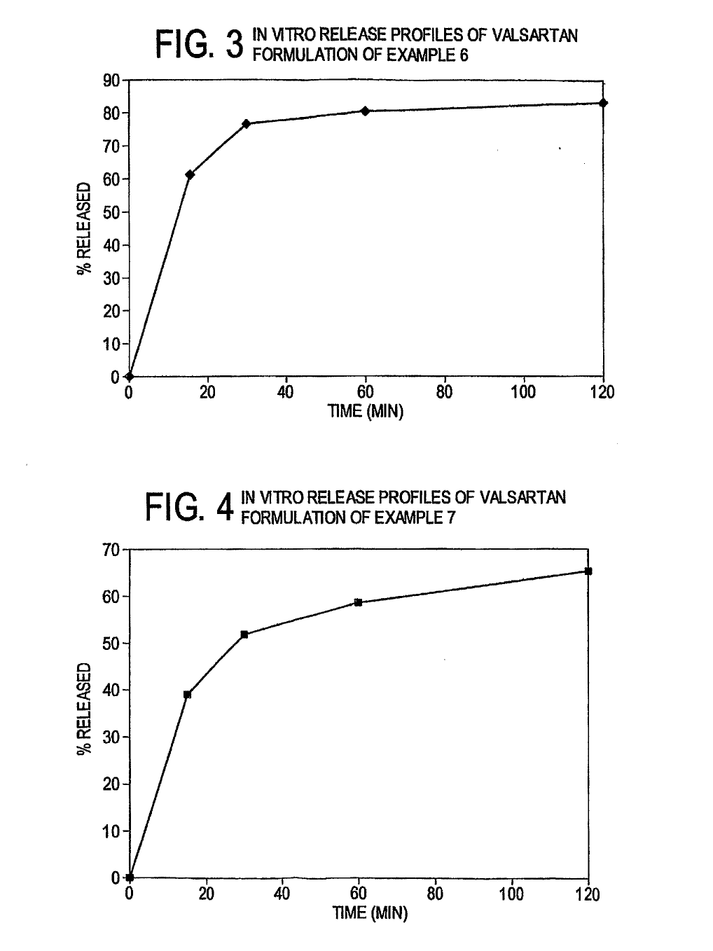

The developed formulation exhibited much higher and much faster drug release profile (FIG. 4) compared to the marketed Diovan tablet, but similar to the exploratory Formulation #1 and slower than the exploratory of Formulation #2.

### Example 8

### Formulation of Solubilized Valsartan into Immediate Release Capsule Dosage Form (80 mg) (Formulation #4) and Comparative Oral Availability Study

TABLE 12
Immediate release capsule formulation
of Valsartan-BEx (Formulation #4)

| Sr. No. | Ingredients | Mg/Capsule |
|---|---|---|
| | Solid dispersion phase | |
| 1. | Valsartan | 80.0 |
| 2. | Vitamin-E TPGS, USP/NF | 40.0 |
| 3. | Poloxamer 407, USP/NF(Lutrol F127) | 40.0 |
| 4. | Microcrystalline Cellulose, USP/NF | 240.0 |
| | (Avicel PH102) | |
| | External | |
| 5. | Microcrystalline Cellulose, USP | 100.00 |
| | (Avicel PH102) | |
| 6. | Cros-povidone, USP (Kollidon.Cl) | 15.0 |
| 7. | Co-Povidone, USP (Kollidon.VA64) | 5.00 |
| 8. | Magnesium Stearate, USP | 6.00 |

### A. Procedure

Vitamin E TPGS and Lutrol 127 MP (Poloxamer 407, USP) were melted together at about 60° C. Valsartan was added to the molten mixture under continuous mixing. Microcrystalline cellulose (Avicel PH102) was added to the mass and cooled to room temperature to produce solid granules.

Microcrystalline cellulose (Avicel PH 102) and a portion of Gros-povidone (KollidonCL) were mixed with the above solid agglomerates. These agglomerates granulated with copovidone dissolved in a mixture of Isopropyl Alcohol: Purified Water (7:3). Wet mass was passed through #12 mesh sieve and dried.

Dried granules were mixed with remaining portion of Cros-povidone (Kollidon.CL), lubricated with Magnesium stearate and filled into capsules.

In-vitro dissolution study: conditions as specified in example 7 were followed.

TABLE 13
Comparative dissolution profile of Formulation
#4 and Diovan in 0.1N HCl

| | Formulation #4 | Diovan ® 80 |
|---|---|---|
| | % Cum. Dissolved | % Cum. Dissolved |
| Time (minutes) | (avg ± S.D.) | (avg ± S.D.) |
| 0 | 0.0 | 0.0 |
| 15 | 70.4 ± 7.0 | 4.1 ± 0.3 |
| 30 | 84.0 ± 3.9 | 10.7 ± 1.4 |
| 60 | 89.5 ± 3.6 | 18.3 ± 0.8 |
| 120 | 92.4 ± 2.8 | 29.4 ± 1.7 |

### Comparative Oral Availability Study

The cross study was designed to evaluate in vivo performance of valsartan capsules of Formulation #4 (T) with respect to Diovan® (R) in healthy male volunteers under fasting condition. Pharmacokinetic parameters, Tₘₐₓ (time to reach maximum drug concentration in blood), Cₘₐₓ (maximum plasma concentration), AUC₀₋ₜ (area under plasma concentration vs. time curve from 0 hours to the time of last sample collected), and AUC_{0-∞} (area under the plasma concentration vs. time curve from 0 hours to infinity (extent of absorption)) were calculated from the data.

TABLE 14
Summary statistics of pharmacokinetic parameters

| | | Untransformed data | | T/R |
|---|---|---|---|---|
| Parameters | Statistics | Reference (R) | Test (T) | ratio |
| Cₘₐₓ (ng/mL) | Mean (C.V. %) | 4405.7 (41.25) | 7215.2 (26.2) | 1.637 |
| AUC₀₋ₜ (hr · ng/mL) | Mean (C.V. %) | 29357.4 (48.9) | 44710.2 (41.6) | 1.523 |
| AUC_{0-∞} (hr · ng/mL) | Mean (C.V. %) | 29907.0 (49.8) | 45484.6 (42.7) | 1.521 |
| Tₘₐₓ (hr) | Median (C.V. %) | 2.66 (37.2) | 1.66 (41.6) | |

The present invention exhibited a higher Cₘₐₓ, AUC₀₋ₜ and, AUC_{0-∞}. compared to the marketed product (FIG. 5). Thus test product was more bioavailable than the marketed product. The test product provided faster onset of action. Also the test product achieved more uniform plasma levels with reduced the variability. A better formulation is thus obtained which reduces variability, possibly allows reduction in dose, and also, gives rapid onset of action which may lead to increased patient compliance.

### Example 9

### pH Independent Dissolution of the Tablet Formulation of the Present Invention (Formulation #5)

TABLE 15
Immediate release formulation of valsartan-BEx, Formulation #5

| Sr. No. | Ingredients | Mg/Tab |
|---|---|---|
| | Solid dispersion phase | |
| 1. | Valsartan | 80.0 |
| 2. | Poloxamer 407, USP/NF (Lutrol F127) | 80.0 |
| 3. | Microcrystalline Cellulose, USP/NF (Avicel PH102) | 240.0 |
| | External | |
| 4. | Microcrystalline Cellulose, USP (Avicel PH102) | 400.00 |
| 5. | Cros-povidone, USP (Kollidon. Cl) | 24.0 |
| 6. | Colloidal Silicon Dioxide, USP (Aerosil 200) | 8.0 |
| 7. | Magnesium Stearate, USP | 8.0 |

### Procedure

Solid dispersion was obtained by mixing the drug with molten Poloxamer 407, USP followed by addition of Microcrystalline cellulose, USP (Avicel PH102) under continuous mixing to the mass and cooling the dispersion to room temperature. The solid dispersion was mixed with weighed quantities of microcrystalline cellulose, USP and Crospovidone followed by lubrication and compression.

### In-Vitro Dissolution Rate Studies

In-vitro dissolution rate studies were carried out in different pH media with following specifications:

### Dissolution Test Apparatus: USP Type II

### Temperature: 37.5±0.5° C.

Dissolution Medium Dissolution studies were carried out in 0.1N HCL, pH 4.5 acetate buffer, and pH 6.8 phosphate buffer USP RPM: 75 (for 0.1N HCl and pH 4.5 buffer) and 50 (for pH 6.8 buffer)
Sampling intervals: 15, 30, 60 and 120 minutes (for 0.1N HCl and pH 4.5 buffer) and 10, 20, 30 and 45 minutes (for pH 6.8 buffer).
Sampling volume: 10 ml

TABLE 16
Multimedia dissolution data

| | 0.1N HCl | | |
|---|---|---|---|
| | (pH~1.2) | pH 4.5 buffer | PH 6.8 buffer |
| | (% Cum. | (% Cum. | (% Cum. |
| Time (minutes) | Dissolved) | Dissolved) | Dissolved) |
| 0 | 0.0 | 0.0 | 0.0 |
| 10 | — | — | 71.7 |
| 15 | 53.1 | 82.7 | — |
| 20 | — | — | 78.6 |
| 30 | 78.8 | 90.7 | 81.9 |
| 45 | — | — | 86.3 |
| 60 | 92.9 | 96.4 | — |

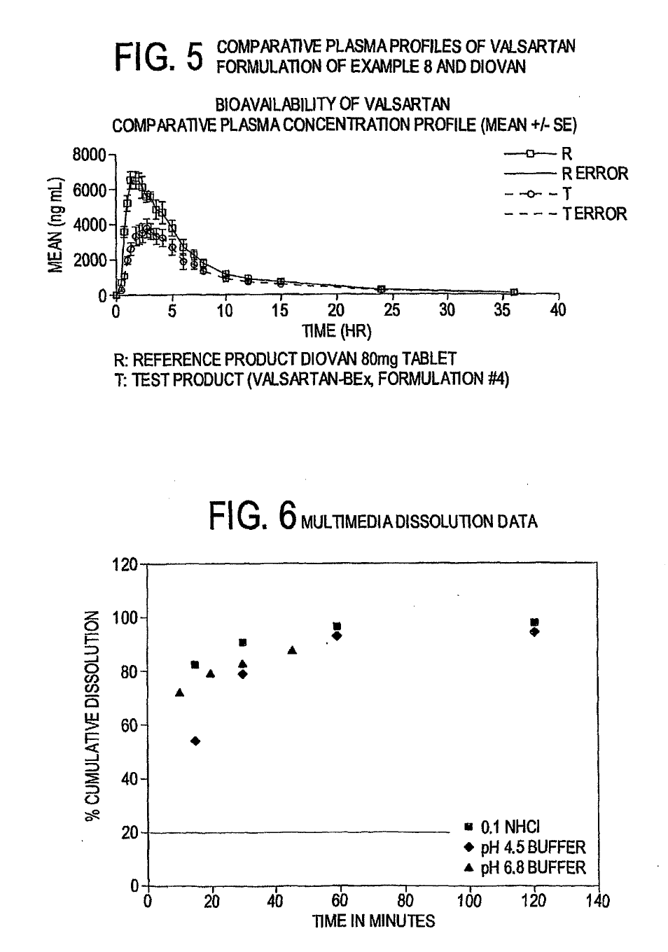

As evident from above table and FIG. 6, the formulation provides pH independent dissolution profile.

### Example 10

### Solubilization of Eprosartan Mesylate

Solid dispersions of eprosartan mesylate with different solubility enhancing agents in different ratios were prepared by mixing drug with solubility enhancing agents or with molten solubility enhancing agents. The solubility of the resulting solid dispersion was determined in 0.1N HCl.

TABLE 17
Solid dispersions of eprosartan mesylate
and solubility enhancement obtained

| | Solubility |
|---|---|
| | in 0.1N HCl |
| Solid dispersion | (mcg/ml) |
| Eprosartan mesylate | 712.0 |
| Eprosartan mesylate:Vitamin E TPGS1:0.2 | 958.3 |
| Eprosartan mesylate:PEG6000, 1:0.5 | 876.9 |
| Eprosartan mesylate:Gelucire 50/13:SLS 1:0.5:0.2 | 1292.4 |
| Eprosartan mesylate:Gelucire 50/13:Gelucire 43/01 | 1003.9 |
| 1:0.5:0.5 | |
| Eprosartan mesylate:Gelucire 50/13:Capmul MCM | 967.8 |
| 1:0.5:0.5 | |
| Eprosartan mesylate:Polyethylene glycol 660 | 1245.6 |
| hydroxystearate, USP (Solutol HS15):SLS 1:1:0.2 | |
| Eprosartan mesylate:Vitamin E TPGS:SLS 1:0.5:0.2 | 1025.9 |

Combination of solubility enhancing agents was found to provide greater solubility enhancement. Addition of SLS was found to dramatically increase the solubility.

### Example 11

### Solubilization of Candesartan Cilexetil

Dispersions of candesartan cilexetil with different excipients were prepared by mixing drug with excipients (e.g. Caprylocaproyl macrogol-8 glycerides, EP (Labrasol) or with molten excipients (e.g. for Solutol HS15). The solubility of the resulting dispersion was determined in 0.1 N HCl.

TABLE 18
Dispersions of candesartan cilexetil
and solubility enhancement obtained

| | Solubility |
|---|---|
| | in 0.1N HCl |
| Solid dispersion | (mcg/ml) |
| Candesartan cilexetil | 0.51 |
| Candesartan cilexetil:Polyethylene glycol 660 | 98.6 |
| hydroxystearate, USP (Solutol HS 15) 1:10 | |
| Candesartan cilexetil:Caprylocaproyl macrogol-8 | 8.9 |
| glycerides, EP (Labrasol) 1:10 | |

Significant increase in solubility was achieved with either Solutol FIS15 or Labrasol.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A composition comprising
valsartan or a salt thereof; and
a solubility enhancing agent.

2. The composition of claim 1, wherein the solubility enhancing agent is selected from the group consisting of surfactant, solubilizer, complexing agent, hydrotropic agent, and cyclodextrin class of molecule.

3. The composition of claim 1, wherein the solubility enhancing agent is selected from the group consisting of PEG-40 hydrogenated castor oil, lauryl macrogol-32 glyceride, stearoyl macrogol glyceride, PEG-20 sorbitan monolaurate, PEG-4 lauryl ether, polyoxyethylene-polyoxypropylene block copolymer, Sodium lauryl sulphate, polyethylene glycol, d-α-tocopheryl polyethylene glycol 1000 succinate, and mixtures thereof.

4. The composition of claim 1, wherein the valsartan is present in an amount of about 1-80% of the composition.

5. The composition of claim 1, wherein the valsartan is present in an amount of about 5-50% of the composition.

6. The composition of claim 1, wherein the valsartan is present in an amount of about 10-30% of the composition.

7. The composition of claim 1, wherein the ratio of the valsartan to the solubility enhancing agent is about 20:1 to about 1:20

8. The composition of claim 1, wherein the ratio of the valsartan to the solubility enhancing agent is about 10:1 to about 1:10.

9. The composition of claim 1, wherein the ratio of the valsartan to the solubility enhancing agent is about 5:1 to about 1:5.

10. The composition of claim 1, further comprising fillers, binders, or lubricants.

11. The composition of claim 1, further comprising microcrystalline cellulose, lactose, calcium silicate, magnesium aluminometasilicate, and mannitol.

12. The composition of claim 1, further comprising a sustained release matrix.

13. The composition of claim 12, wherein the sustained release matrix is selected from the group consisting of polyalkylene oxides, cellulosic polymers, acrylic acid, methacrylic acid polymers, esters of acrylic acid and methacrylic acid polymers, maleic anhydride polymers, polymaleic acid, poly (acrylamides); poly(olefinic alcohol)s, poly(N-vinyl lactams), polyols, polyoxyethylated saccharides, polyoxazolines, polyvinylamines, polyvinylacetates, polyimines, starch, starch-based polymers, polyurethane hydrogels, chitosan, polysaccharide gums, zein, shellac-based polymers, polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch, polyvinyl alcohol, copolymers, and mixtures thereof.

14. The composition of claim 1, wherein the composition is a tablet or a capsule.

15. The composition of claim 1, wherein dissolution at pH<3 is at least 40% in 60 minutes.

16. The composition in claim 1 where the dissolution in 0.1 N HCl should be at least 20% in 5 minutes, 25% in 15 minutes, 30% in 30 minutes, 35% in 45 minutes and 40% in 60 minutes.

17. The composition in claim 1, where the dissolution is substantially independent of pH.

18. The composition in claim 1, wherein Tₘₐₓ is about 1-2 hours.

19. The composition in claim 1, wherein Tₘₐₓ, is about 1-2 hours and Cₘₐₓ is at least 80 ng/ml per mg of valsartan dose.

20. The composition in claim 1, wherein Tₘₐₓ is about 1-2 hours and AUC is at least 500 ng·hr/ml per mg of valsartan dose.

21. The composition in claim 1, wherein the coefficient of variation (CV) is no more than 45% for AUC.

22. The composition in claim 1, wherein the coefficient of variation (CV) is no more than 40% for AUC

23. The composition in claim 1, wherein the coefficient of variation (CV) is no more than 35% for Cₘₐₓ.

24. The composition in claim 1, wherein the coefficient of variation (CV) is no more than 30% for Cₘₐₓ.

25. The composition of claim 1, further comprising another anti-hypertensive agent, an anti-obesity agent, an anti-diabetic agent, a beta-blocker, an inotropic agent, a hypolipidemic agent, or combinations thereof.

26. The composition of claim 25, wherein the other anti-hypertensive agent is selected from the group consisting of HCTZ, calcium blockers, beta-blockers, ACE inhibitors, inotropic agents, hypolipidemic agents, renin inhibitor, and combinations thereof.

27. A method for making a valsartan composition, for increasing the solubility of valsartan, for increasing the absorption of valsartan, or for reducing inter- or intra-patient absorption variability of valsartan, said method comprising the steps of
(a) providing valsartan or a salt thereof;
(b) providing a solubility enhancing agent; and
(c) mixing the valsartan with the solubility enhancing agent.

28. The method of claim 27, wherein step (c) involves melt granulation, intimate physical mixing, spray drying, or solvent evaporation.

29. The method of claim 27, wherein the solubility enhancing agent is selected from the group consisting of surfactant, solubilizer, complexing agent, hydrotropic agent, and cyclodextrin.

30. The method of claim 27, wherein the solubility enhancing agent is selected from the group consisting of PEG-40 hydrogenated castor oil, lauryl macrogol-32 glyceride, stearoyl macrogol glyceride, PEG-20 sorbitan monolaurate, PEG-4 lauryl ether, polyoxyethylene-polyoxypropylene block copolymer, Sodium lauryl sulphate, polyethylene glycol, d-α-tocopheryl polyethylene glycol 1000 succinate, and mixtures thereof.

31. The method of claim 27, wherein the ratio of the valsartan to the solubility enhancing agent is about 10:1 to about 1:10.

32. The method of claim 27, wherein the ratio of the valsartan to the solubility enhancing agent is about 5:1 to about 1:5.

33. The method of claim 27, further comprising the step of adding fillers, binders, or lubricants.

34. The method of claim 27, further comprising the step of adding microcrystalline cellulose, lactose, calcium silicate, magnesium aluminometasilicate, or mannitol.

35. The method of claim 27, further comprising the step of adding a sustain released matrix.

36. The method of claim 35, wherein the sustained release matrix is selected from the group consisting of polyalkylene oxides, cellulosic polymers, acrylic acid, methacrylic acid polymers, esters of acrylic acid and methacrylic acid polymers, maleic anhydride polymers, polymaleic acid, poly (acrylamides); poly(olefinic alcohols, poly(N-vinyl lactams), polyols, polyoxyethylated saccharides, polyoxazolines, polyvinylamines, polyvinylacetates, polyimines, starch, starch-based polymers, polyurethane hydrogels, chitosan, polysaccharide gums, zein, shellac-based polymers, polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch, polyvinyl alcohol, copolymers, and mixtures thereof.

37. The method of claim 27, further comprising the step of tableting the mixture.

38. The method of claim 27, further comprising the step of adding another anti-hypertensive agent, an anti-obesity agent, an anti-diabetic agent, a beta-blocker, an inotropic agent, a hypolipidemic agent, or combinations thereof.

39. The method of claim 38, wherein the other anti-hypertensive agent is selected from the group consisting of HCTZ, calcium blockers, beta-blockers, ACE inhibitors, inotropic agents, hypolipidemic agents, renin inhibitors, and combinations thereof.

40. The method for treating a disease selected from the group consisting of circulatory disease, kidney disease, cerebral dysfunction, diabetic complications, arteriosclerosis, hyperaldosteronism, multiple system organ failure, scleroderma, anxiety neuroses, catatonia, and dyspepsia; said method comprising the step of administering to a mammal in need thereof a therapeutically effective amount of the composition of claim 1.

41. The method of claim 40, wherein the circulatory disease is hypertension, cardiac disease, or peripheral circulatory insufficiency.

42. The method of claim 40, wherein the kidney disease is. glomerulonephritis or renal insufficiency.

43. The method of claim 40, wherein the cerebral dysfunction is stroke, Alzheimer's disease, depression, amnesia, or dementia.

44. The method of claim 40, wherein the diabetic complications is retinopathy or nephropathy.

45. The method of claim 40, wherein the arteriosclerosis is manifested by hypertension, stroke, heart attack, angina, or ischemia of ischemia of gastrointestinal tract or extremities.

46. The method of claim 40, wherein the composition further comprising another anti-hypertensive agent, an anti-obesity agent, an anti-diabetic agent, a beta-blocker, an inotropic agent, a hypolipidemic agent, or combinations thereof.

47. The composition of claim 46, wherein the other anti-hypertensive agent is selected from the group consisting of HCTZ, calcium blockers, beta-blockers, ACE inhibitors, inotropic agents, hypolipidemic agents, renin inhibitors, and combinations thereof.

48. A composition comprising
an Angiotensin II receptor blocker (ARB) or a salt thereof; and
a solubility enhancing agent.

49. The composition of claim 48, wherein the solubility enhancing agent is selected from the group consisting of surfactant, solubilizer, complexing agent, hydrotropic agent, and cyclodextrin class of molecule.

50. The composition of claim 48, wherein the solubility enhancing agent is selected from the group consisting of PEG-40 hydrogenated castor oil, lauryl macrogol-32 glyceride, stearoyl macrogol glyceride, PEG-20 sorbitan monolaurate, PEG-4 lauryl ether, polyoxyethylene-polyoxypropylene block copolymer, Sodium lauryl sulphate, polyethylene glycol, d-α-tocopheryl polyethylene glycol 1000 succinate, and mixtures thereof.

51. The composition of claim 48, wherein the ARB is present in an amount of about 1-80% of the composition.

52. The composition of claim 48, wherein the ARB is present in an amount of about 5-50% of the composition.

53. The composition of claim 48, wherein the ARB is present in an amount of about 10-30% of the composition.

54. The composition of claim 48, wherein the ratio of the ARB to the solubility enhancing agent is about 20:1 to about 1:20

55. The composition of claim 48, wherein the ratio of the ARB to the solubility enhancing agent is about 10:1 to about 1:10.

56. The composition of claim 48, wherein the ratio of the ARB to the solubility enhancing agent is about 5:1 to about 1:5.

57. The composition of claim 48, further comprising fillers, binders, or lubricants.

58. The composition of claim 48, further comprising microcrystalline cellulose, lactose, calcium silicate, magnesium aluminometasilicate, and mannitol.

59. The composition of claim 48, further comprising a sustained release matrix.

60. The composition of claim 59, wherein the sustained release matrix is selected from the group consisting of polyalkylene oxides, cellulosic polymers, acrylic acid, methacrylic acid polymers, esters of acrylic acid and methacrylic acid polymers, maleic anhydride polymers, polymaleic acid, poly (acrylamides); poly(olefinic alcohol)s, poly(N-vinyl lactams), polyols, polyoxyethylated saccharides, polyoxazolines, polyvinylamines, polyvinylacetates, polyimines, starch, starch-based polymers, polyurethane hydrogels, chitosan, polysaccharide gums, zein, shellac-based polymers, polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch, polyvinyl alcohol, copolymers, and mixtures thereof.

61. The composition of claim 48, wherein the composition is a tablet or a capsule.

62. The composition of claim 48, wherein the ARB is selected from the group consisting of candesartan, eprosartan, irbesartan, losartan, olmesartan, telmisartan, valsartan, and pratosartan.

63. The composition of claim 48, further comprising another anti-hypertensive agent, an anti-obesity agent, an anti-diabetic agent, a beta-blocker, an inotropic agent, a hypolipidemic agent, or combinations thereof.

64. The composition of claim 63, wherein the other anti-hypertensive agent is selected from the group consisting of HCTZ, calcium blockers, beta-blockers, ACE inhibitors, inotropic agents, hypolipidemic agents, and combinations thereof.

65. The composition of claim 48, wherein the oral availability of an ARB is 1.2-4 times higher than that of the ARB by itself.
